(19) United States
(12) Patent Application Publication
(10) Pub. No.: US 2025/0009583 A1
(43) Pub. Date: Jan. 9, 2025

(54) INTEGRATED MONITORING SYSTEMS, DEVICES, AND METHODS FOR IMPROVED INCUBATOR TREATMENTS

(71) Applicants: Loma Linda University Health, Loma Linda, CA (US); The Regents of the University of California, Oakland, CA (US); University of Notre Dame, South Bend, IN (US)

(72) Inventors: Christopher G. Wilson, Loma Linda, CA (US); Nosang V. Myung, South Bend, IN (US); Thomas F. Stahovich, Riverside, CA (US)

(21) Appl. No.: 18/762,867

(22) Filed: Jul. 3, 2024

Related U.S. Application Data
(60) Provisional application No. 63/512,334, filed on Jul. 7, 2023.

(51) Int. Cl. A61G 11/00; A61B 5/00; A61B 5/0205; A61B 7/04; G16H 40/67; G16H 50/70

(52) U.S. Cl. CPC A61G 11/00; A61B 5/0022; A61B 5/0205; A61B 5/7267; G16H 40/67; G16H 50/70; A61B 5/0077; A61B 7/04; A61B 2503/045

(57) ABSTRACT

Provided herein are methods, systems, and devices for integrated monitoring of patients, such as infants, within incubators. In an embodiment, an integrated monitoring system may include an integrated monitoring device communicatively coupled to a plurality of sensors. The integrated monitoring device may include a processor that executes machine-readable instructions to receive an environmental condition of an incubator and a clinical variable of the infant from the plurality of sensors. The processor may generate a predicted outcome based on application of the environmental condition and the clinical variable to a model configured to associate sensor data with clinical outcomes. In response to the predicted outcome being outside a threshold, the processor may output a notification to a user device communicatively coupled to the integrated monitoring device.

### CROSS-REFERENCE TO RELATED APPLICATION

This application claims priority to U.S. Provisional Application No. 63/512,334 that was filed on Jul. 7, 2023. The entire content of the applications referenced above is hereby incorporated by reference herein.

### TECHNICAL FIELD

The present disclosure relates to integrated monitoring systems and, more particularly, to systems, devices, and methods of integrated monitoring for observing or treating an infant in an incubator.

### BACKGROUND

Infant incubators, such as the Isolette® incubator, are specialized cribs systems for providing care to infants. For example, incubators may be located in hospital rooms, nurseries, or neonatal intensive care units to offer an individual, temperature-controlled space for each newborn. Each incubator may generally have a base or mattress on which a newborn is placed, a heater that warms the interior of the incubator, and a cover that protects the newborn from a surrounding environment. The cover may be a clear barrier with access ports to allow medical providers or family members to contact and care for the newborn. Certain incubators may also include a 20) limited sensing capacity, such as a thermometer to sense the temperature therein. By providing a safe and warm area, these existing incubators meet the basic needs of infants being monitored in a medical setting. The infants may therefore be nurtured in the incubators until they are capable of maintaining homeostasis in a less controlled environment.

In some situations, infants may be born with or develop health symptoms that are monitored and treated during their stay in incubators at a medical center. For example, certain pre-mature infants may receive intensive, around-the-clock care within incubators to ensure their appropriate growth and development. Medical providers may also remedy or address any illnesses, diseases, or symptoms displayed by other infants in incubators. As such, the medical providers may observe multiple variables of the infants to manually monitor the health of each infant, of which there might be dozens within a single hospital. However, there is a significant lack of integrated monitoring features in the incubators, necessitating time-intensive analysis for each individual. Moreover, some infants may develop symptoms or conditions that could have been avoided if earlier intervention was provided or if earlier indications were recognized.

Accordingly, there is a demand for improved systems and methods that optimize medical care provided to infants, beyond current capabilities.

### SUMMARY

Computer-implemented methods of integrated monitoring systems for supporting an infant in an incubator are disclosed. Non-transitory computer-readable media, devices, and systems for performing methods of the present disclosure are also disclosed.

In some aspects, the present disclosure provides an integrated monitoring system for supporting an infant in an incubator. The integrated monitoring system also includes a plurality of sensors configured to sense at least one environmental condition of the incubator and at least one clinical variable of the infant. The integrated monitoring system also includes an integrated monitoring device communicatively coupled to the plurality of sensors. The integrated monitoring device may include a processor and a non-transitory machine-readable storage medium storing processor-executable instructions that, when executed by the processor, cause the processor to: receive the at least one environmental condition of the incubator and the at least one clinical variable of the infant from the plurality of sensors: generate a predicted outcome based on application of the at least one environmental condition of the incubator and the at least one clinical variable of the infant to a model configured to associate sensor data with clinical outcomes; and in response to the predicted outcome being outside a threshold, output a notification to a user device communicatively coupled to the integrated monitoring device. In some aspects, the present disclosure provides an integrated monitoring device for supporting a patient in an incubator. The integrated monitoring device also includes a microphone configured to collect audio data associated with the incubator. The integrated monitoring device also includes a camera configured to collect video data associated with the incubator. The integrated monitoring device also includes a plurality of input/output ports configured to connect to a plurality of sensors to collect sensor data associated with the incubator. The integrated monitoring device also includes a controller may include a processor and a non-transitory machine-readable storage medium storing processor-executable instructions that, when executed by the processor, cause the processor to: receive the audio data, the video data, and the sensor data; combine the audio data, the video data, and the sensor data into a stream of multi-dimensional sensor data; process the stream of multi-dimensional sensor data through a sensor-outcome model configured to associate past sensor data with clinical outcomes to generate a predicted outcome; and in response to determining that the predicted outcome is outside a threshold, output a suggested treatment adjustment to a user device.

In some aspects, the present disclosure provides a method of providing clinical decision support for monitoring a patient of an incubator, including receiving, via a processor of an integrated monitoring device: audio data from a microphone of the integrated monitoring device; video data from a camera of the integrated monitoring device; and sensor data from a plurality of sensors associated with the integrated monitoring device. The method also includes combining, via the processor, the audio data, the video data, and the sensor data into a stream of current multi-dimensional sensor data; processing, via the processor, the stream of current multi-dimensional sensor data through a sensor-outcome model to generate a predicted outcome; and in response to a determination that the predicted outcome is outside a threshold, outputting a suggested treatment adjustment to a user device.

Other aspects and features of the present disclosure will become apparent to those of ordinary skill in the art after reading the detailed description herein and the accompanying figures.

### BRIEF DESCRIPTION OF THE DRAWINGS

Embodiments described herein are illustrated by examples and not limitations in the accompanying drawings, in which like references indicate similar features. Furthermore, in the drawings some conventional details have been omitted so as not to obscure the inventive concepts described herein.

FIG. 1 is a block diagram of an integrated monitoring system for use with an incubator, according to an embodiment of the present disclosure.

FIG. 2 is a block diagram of an integrated monitoring system for use with multiple incubators, according to an embodiment of the present disclosure.

FIG. 3 is a block diagram of an integrated monitoring device communicatively coupled to various sensors and components associated with an incubator, according to an embodiment of the present disclosure.

FIG. 4 is a block diagram of a controller of an integrated monitoring device receiving data streams to generate a predicted outcome based on a model, according to an embodiment of the present disclosure.

FIG. 5 is a flowchart of a method to use an integrated monitoring system to provide clinical decision support, according to an embodiment of the present disclosure.

FIG. 6 is a flowchart of a method to train a sensor-outcome model of an integrated monitoring system based on multi-dimensional sensor data, according to an embodiment of the present disclosure.

FIG. 7 is a block diagram of an integrated monitoring system having a modular system, according to an embodiment of the present disclosure.

While the disclosure will be described in connection with the preferred embodiments, it will be understood that it is not intended to limit the disclosure to those embodiments. On the contrary, it is intended to cover all alternatives, modifications, and equivalents, as may be included within the spirit and scope of the disclosure as defined by the appended claims.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described. To facilitate understanding, certain non-limiting definitions of terms are provided herein. As used herein, a “subject” refers to an animal, such as a mammal, including a primate (such as a human or a non-human primate) and a non-primate (such as a mouse or a bird). In some aspects of the present disclosure, the subject is a human. In some aspects, the subject is a pediatric subject, such as a neonate, an infant, or a child. In other aspects, the subject is an adult subject. As used herein, a “patient” refers to a subject who shows symptoms and/or signs of a disease, is under treatment for disease, has been diagnosed with a disease, and/or is at risk of developing a disease. A “patient” can include human or veterinary subjects. Any reference to a subject in the present disclosure should be understood to include the possibility that the subject is a “patient” unless clearly dictated otherwise by context. More specifically, the subject in certain aspects is a patient that has been placed or directed into an incubator for health monitoring. As used herein, a “user device” refers to an electronic device that can receive information from another electronic device and present information to a user of the user device. The user device may include any suitable electronic device having a communicating feature, such as a wireless connection component, and internet connection component, and so forth. In certain embodiments, the user device is a cellular telephone, a smart phone, a smart watch, a pair of smart glasses, a tablet, a laptop, a desktop, and so forth.

As introduced above, an infant incubator may be equipped with a heating component and a cover or protective shield that isolates a warm chamber for infants from a surrounding environment, such as a hospital unit, nursery, or room. As will be understood, it is particularly desirable to provide systems, devices, and methods for integrated, effective, and reliable monitoring of infants within incubators. Indeed, there exists a need for intelligent incubators that streamline and improve the clinical outcomes of the infants being treated therein. The integrated monitoring system disclosed herein may guide care of infants based on leveraging a multitude of real-time sensor data streams with one or more artificial intelligence (AI) models to predictively direct patient health toward optimal outcomes. For example, the integrated monitoring system may incorporate multiple sensors, including sensors for measuring temperature, humidity, pressure, volatile organic compound (VOC) concentration, oxygen concentration, and carbon dioxide concentration, in addition to sensors for recording video, audio, and electrocardiogram (EKG) data. By simultaneously measuring a wide variety of environmental conditions and clinical variables in real-time, the integrated monitoring system is able to output suitable alerts and suggested treatment adjustments for medical providers to maintain or improve the health of infants in incubators more effectively. As used herein, an “environmental condition” refers to any measurable parameter or level associated with the environment within the incubator, such as the unoccupied volume of the chamber therein. As used herein, a “clinical variable” refers to any measurable parameter or level associated with the occupant of the incubator. The sensors and components disclosed herein may each be used to monitor one or more environmental conditions of the incubator, one or more clinical variables of the infant, or both.

Furthermore, by recording the sensor data and associated patient outcomes, the integrated monitoring system may produce datasets for further training of the AI models. The integrated monitoring system may leverage the continuously updated AI models to provide decision support to medical providers in providing optimal treatment plans and recognizing early signs of potential complications. In certain cases, the integrated monitoring system may automatically regulate incubator conditions and set points to ensure optimal outcomes for the infants. As such, the integrated monitoring system and AI models thereof are beneficial for improving patient outcomes and reducing certain complications, such as sepsis or retinopathy of prematurity. For example, there is currently no optimal strategy for managing oxygen levels for premature infants. High oxygen levels may be provided to an incubator when breathing issues such as apnea are a concern, but an excess of oxygen may lead to retinopathy of prematurity in certain cases. The data collected by the integrated monitoring system enables the creation and tailoring of AI models for optimally regulating oxygen levels, while maintaining other operating conditions in desired ranges, thus minimizing both the occurrence and degree of retinopathy and other undesired conditions.

As a non-limiting example, the integrated monitoring system of certain embodiments may include an integrated monitoring device that may include a single-board computer with attached video camera and microphone. Communicatively coupled to the integrated monitoring device, the integrated monitoring system may include a miniature gas sensor board with multiple gas sensing elements as well as temperature, pressure, gas flow; and/or humidity sensors. The collected sensor data can be streamed in real time over a wired connection (such as via ethernet) and/or wireless communication protocols to be viewed remotely. As further examples, the wide variety of monitored environmental conditions and clinical variables may include any suitable combination of skin color (via video data), activity levels (via audio and video data), VOC emissions from metabolic processes (via gas sensor data), oxygen and carbon dioxide concentration (via gas sensor data), and environmental and body temperature (via temperature sensor data). Moreover, in certain embodiments, the integrated monitoring system or integrated monitoring device may include a distributed or modular architecture, having a sensor control module, an analysis module, a communication module, and/or an actuation module that cooperate to perform the techniques disclosed herein. As will be understood, the integrated monitoring system thus empowers physicians or clinicians to better monitor and treat infants within incubators.

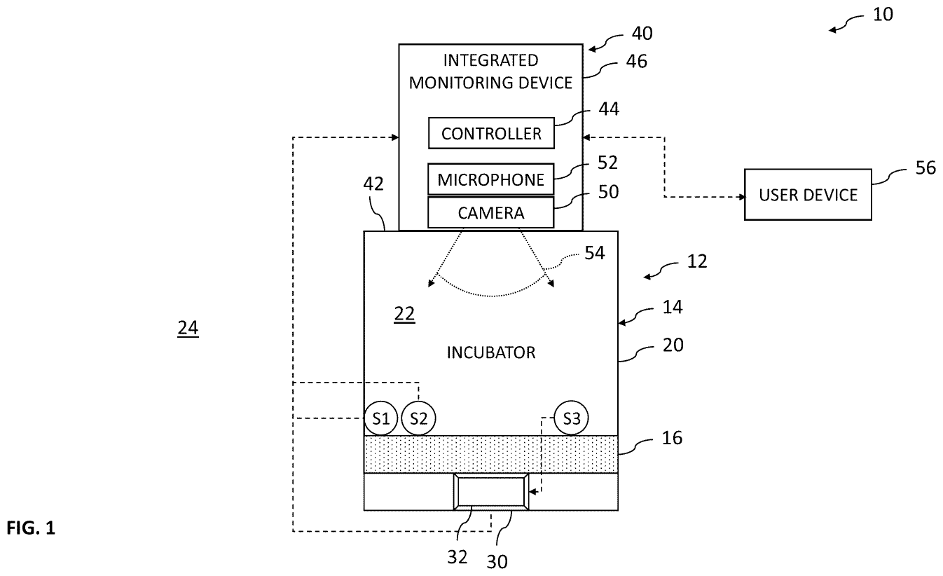

FIG. 1 illustrates an example of an integrated monitoring system 10, in accordance with aspects of the present disclosure. As will be understood, the integrated monitoring system 10 may be implemented with or coupled to an incubator 12 to provide integrated, effective, and reliable monitoring of an infant associated with the incubator 12. In certain embodiments, the incubator 12 includes a main housing 14 that has a mattress 16 or base to provide a comfortable and secure resting surface for the infant. A cover 20 of the main housing 14 may be disposed over the mattress 16 to isolate a chamber 22 of the incubator 12 from a surrounding environment 24. The cover 20 may be any suitable rigid material, such as a translucent acrylic or plastic material that enables viewing and monitoring of the infant within the incubator 12. As illustrated, the incubator 12 of certain embodiments includes an incubator monitor 30 that is physically coupled to the main housing 14. In other embodiments, the incubator monitor 30 may be coupled to a separate stand or support, instead of the main housing 14 of the incubator 12. The incubator monitor 30 may be a medical device that is designed to collect and present various data regarding the care for and health of the infant. The incubator monitor 30 may therefore include a display 32 that presents visualizations of the collected data. Some embodiments of the incubator monitor 30 may also include a speaker component for emitting audio alerts regarding the infant.

The integrated monitoring system 10 of the present embodiment includes an integrated monitoring device 40 that is disposed on an upper surface 42 of the cover 20. The integrated monitoring device 40 includes a controller 44, the operation of which will be described in detail below. In general, the controller 44 collects and analyzes streams of real-time data from various sensors associated with the incubator 12 to facilitate monitoring and treatment of the infant. The integrated monitoring device 40 may include a housing 46 or frame that retains the controller 44, a camera 50), and a microphone 52 to the upper surface 42 of the cover 20. In certain embodiments, the housing 46 is physically coupled to the cover 20, such as via an adhesive, a mechanical fastener, a hook-and-loop connection, or any other suitable attachment device. The housing 46 may be removably attached to the cover 20 to allow the integrated monitoring device 40 to be moved to different incubators, in some embodiments. In certain embodiments, the housing 46 may be placed on top of the cover 20 and remain there via friction, without adding coupling mechanisms. Although illustrated and discussed with respect to the integrated monitoring device 40 being above the infant, other embodiments may include the integrated monitoring device 40 placed near or coupled to a lateral side of the cover 20. From the position of the camera 50 above or otherwise in view of the infant, the camera 50 or any suitable lens of the camera 50 may have a field of view 54 that encompasses the mattress 16 and any infant thereon. The camera 50 of the integrated monitoring device 40 may therefore capture image data or video data of the infant within the chamber 22 and transfer the data to the controller 44. Moreover, the microphone 52 may capture audio data of any noises or sounds from within the chamber 22 of the incubator 12 or the surrounding environment 24 and transfer the audio data to the controller 44. In some embodiments, the data from the camera 50) and/or the microphone 52 may be used to determine an activity level of the infant.

The integrated monitoring system 10 additionally includes other sensors positioned to monitor environmental conditions within or around the incubator 12 and/or clinical variables of the infant within the incubator 12. As one example, the integrated monitoring system 10 may include a first sensor S1 and a second sensor S2 communicatively coupled to the integrated 30) monitoring device 40. The first sensor S1 may monitor one or more environmental conditions of the incubator 12, such as a particular gas composition, an environmental temperature, a humidity, a flowrate, and/or a pressure. The second sensor S2 may monitor one or more clinical variables of the infant, such as a temperature, a blood measurement, and/or a heart measurement.

In other embodiments, the second sensor S2 may monitor additional environmental conditions of the incubator 12. Further examples of sensors and the corresponding real-time sensor data are discussed with reference to subsequent figures. In some embodiments, one or more of the sensors S1. S2 are wireless transducers that transmit sensor data to the integrated monitoring device 40) via a suitable wireless communication protocol. In other embodiments, one or more of the sensors S1. S2 are wired transducers that are coupled to the integrated monitoring device 40) via at least one communication cable. In such embodiments, the communication cable may be extended from the housing 46 of the integrated monitoring device 40, through a port or aperture of the cover 20, and to a sensing portion of the sensors S1, S2. In certain embodiments, two or more sensors, such as the sensors S1, S2, may be coupled to a single communication cable, thus streamlining deployment of the integrated monitoring system 10 for the incubator 12. In the illustrated embodiment, the incubator monitor 30 is communicatively coupled to the integrated monitoring device 40. The incubator monitor 30 may therefore relay or pass along various received sensor data to the integrated monitoring device 40 for further analysis. For example, the integrated monitoring system 10 may include a third sensor S3 that is communicatively coupled to the incubator monitor 30. Environmental conditions and/or clinical variables monitored by the third sensor S3 may therefore be transmitted to the incubator monitor 30, which then transmits or relays the data to the integrated monitoring device 40) as a continuous data stream in real time. In certain embodiments, the communication between the 20) third sensor S3 and the incubator monitor 30 or between the incubator monitor 30 and the integrated monitoring device 40 may be wireless or wired.

The integrated monitoring system 10 may further include one or more user device 56 that is communicatively coupled to the integrated monitoring device 40. During the course of continuous monitoring, the integrated monitoring device 40 may transmit alerts, status updates, and/or suggested treatment adjustments to the user device 56 to inform medical providers or family members of the progress of the infant in the incubator 12. The user device 56 may be any suitable electronic device having at least one output device for providing data to a person, such as a display, a speaker, and/or a haptic output device. As non-limiting examples, the user device 56 may be a mobile cellular device, a smart watch, a tablet, a laptop, or a computer station. In any case, in response to the integrated monitoring device 40 analyzing data from the sensors S1, S2, S3 and/or incubator monitor 30, the integrated monitoring device 40 may transmit information regarding the infant to the user device 56. In some embodiments, the integrated monitoring device 40 may transmit information to the user device 56 at regular intervals and/or in response to receiving an information request from the user device 56. The integrated monitoring system 10 may therefore support provision of more effective, reliable, and in-depth information to guide care of the infant than was previously possible before the present techniques.

In certain embodiments, the integrated monitoring system 10 may also monitor or control operation of various subsystems associated with the incubator 12. For example, the incubator may be provided with a heating component, a gas flow controller, an oxygen source, and/or a ventilator, in some embodiments. In such cases, the integrated monitoring device 40 may transmit control signals to one or more of the subsystems to coordinate their operation for providing optimal patient treatments. In certain embodiments, the integrated monitoring device 40) may transmit a request to the user device 56 for user approval of any treatment adjustments. In response to or after receiving an indication of user approval, the integrated monitoring device 40 may therefore provide control signals to the subsystems to deliver optimal treatment outcomes for the patient, thus freeing limited healthcare provider time for utilization on other responsibilities.

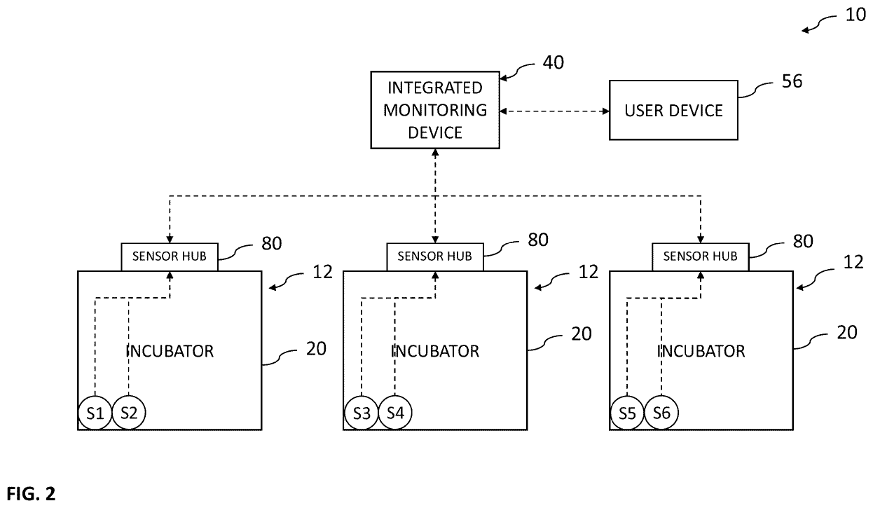

FIG. 2 illustrates another example of the integrated monitoring system 10, in accordance with aspects of the present disclosure. In the illustrated embodiment, the integrated monitoring system 10 may be implemented with or coupled to multiple incubators 12 to provide integrated, effective, and reliable monitoring of the infants therein. For example, the integrated monitoring system 10 of certain embodiments may include a distributed sensing array, such as via multiple sensor hubs 80 that are each disposed on or adjacent to the cover 20 of a respective incubator 12. The sensor hubs 80 may each include a camera and a microphone, in certain embodiments. Moreover, sensors S1-S6 may be distributed throughout the incubators 12 to sense and collect real-time data therefrom. The sensors S1-S6 may transmit the data to the sensor hubs 80, which may forward the data to the integrated monitoring device 40 for primary data analysis. The integrated monitoring device 40 may therefore operate as a primary controller that receives sensor data representing a holistic picture of the treatments of multiple infants and generates alerts or suggested treatment adjustments to optimize the treatments. As discussed herein, the integrated monitoring device 40 may forward the alerts or the suggested treatment adjustments to the user device 56, or multiple user devices 56, to maintain or improve the health of infants within the incubators 12.

In some embodiments, the integrated monitoring system 10 may have a modular control arrangement, where the sensor hubs 80 may be integrated monitoring devices that perform local analysis for a single incubator 12. The sensor hubs 80 may then be optionally connected to a supervisory controller, such as the illustrated integrated monitoring device 40, that monitors operation of multiple integrated monitoring devices and associated incubators 12. The supervisory controller may perform more global monitoring and evaluate potential environmental adjustments, including adjustments to light levels, noise levels, room temperature, and so on. For example, if multiple infants begin displaying symptoms of being too cool or too warm, the supervisory controller may present recommendations to a technician or directly instruct a thermostat to adjust a temperature of the room. As such, multiple infants can reach a comfortable homeostasis more effectively in the room having an adjusted temperature.

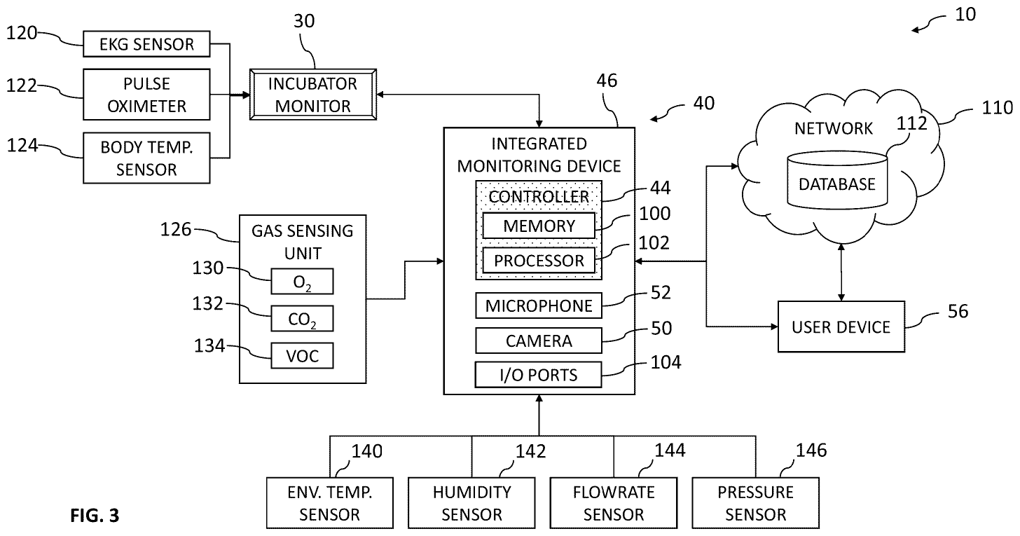

FIG. 3 illustrates an example block diagram of the integrated monitoring system 10, including the integrated monitoring device 40 device communicatively coupled to various non-limiting sensors and components associated with an incubator, in accordance with aspects of the present disclosure. As discussed above, the integrated monitoring device 40 may be coupled to, disposed on, or positioned adjacent to the incubator, such that the microphone 52 and the camera 50 thereof may generate audio and/or visual signals associated with a current physical state and care of the infant. The microphone 52 and the camera 50 may be coupled to the housing 46 or frame of the integrated monitoring device 40, which may also support the controller 44. The controller may include a memory 100 and a processor 102. The memory 100 may include or store instructions executable by the processor 102.

As used herein, a “processor”, processing resource, or processing circuitry may be a plurality of processors connected together in communication with an electronic communications network. In other embodiments, the processors may be a group of graphical processing units configured to work in parallel as a GPU cluster. A processor may include a single processor device and/or a plurality of processor devices, such as distributed processors. Additionally, a processor may be any suitable processor capable of executing/performing instructions. For example, a processor may include a central processing unit (CPU), a semiconductor-based microprocessor, a graphics processing unit (GPU), a field-programmable gate array (FPGA) to retrieve and execute instructions, and/or a real-time processor (RTP) that carries out program instructions to perform the basic arithmetical, logical, and input/output operations required to support the integrated 30) monitoring system 10. A processor may include code, such as processor firmware, a protocol stack, a database management system, an operating system, or a combination thereof, that creates an execution environment for program instructions. Processes and logic flows described herein may be performed by one or more programmable processors executing one or more computer programs to perform functions by operating on input data and generating corresponding output.

In an example, the memory 100 may be a non-transitory machine-readable storage medium. As used herein, a “machine-readable storage medium” may be any electronic, magnetic, optical, or other physical storage apparatus or cyber-physical separation storage to contain or store information such as executable instructions, data, and the like. For example, any machine-readable storage medium described herein may be any of random-access memory (RAM), volatile memory, non-volatile memory, flash memory; a storage drive such as hard drive, a solid-state drive, any type of storage disc, and the like, or a combination thereof. As noted, the memory 100 may store or include instructions executable by the processor 102. Accordingly, the processor 102 may execute machine-readable instructions of the memory 100 to monitor and analyze the health of the infant in the incubator in real time, as discussed in more detail below: The integrated monitoring device 40 may include any suitable number of input and/or output (I/O) ports 104 for enabling communication between other components of the integrated monitoring system 10 and the integrated monitoring device 40, such as via hard wiring the components together. In some embodiments, the integrated monitoring device 40 may also include wireless communication components that facilitate wireless communication between various components. For example, wireless communication may be Wi-Fi®, Bluetooth®, ZigBee, or forms of near field communications. In some embodiments, the user device 56 of the integrated monitoring system 10 may be communicatively coupled to the integrated monitoring device 40 via a network 110, such as a cloud network. The network 110 may host a database 112, in certain embodiments, which may store a collection of data associating various clinical variables and environmental conditions with medical outcomes or diagnoses. From this database 112, the integrated monitoring device 40 may create and/or train AI models to guide future evaluations of infants more efficiently. In other embodiments, the database 112 may be stored locally within the memory 100 of the controller 44.

As illustrated, the integrated monitoring system 10 may include a wide variety of sensors that monitor an extensive range of environmental conditions of the surrounding environment and clinical variables of the infant. Various non-limiting examples of the environmental conditions 30) and clinical variables contemplated by the present disclosure are provided below; along with an example of an associated sensor for measuring the respective environmental conditions and clinical variables. The sensors of the integrated monitoring system 10 may monitor and relay data at an increased resolution, compared to previous incubator sensors that may only activate based on user input, or when a high-risk event is occurring. For example, the sensors disclosed herein may transmit sensor data multiple times per second, once per second, once per 10 seconds, or at any other suitable rate that provides a comprehensive view of the health of the infant and the surrounding environment.

With initial attention to the clinical variables, the incubator monitor 30 introduced above may be communicatively coupled to the integrated monitoring device 40 to relay data thereto. For example, the integrated monitoring system 10 may include an electrocardiogram sensor 120) communicatively coupled to the incubator monitor 30 to transmit data of a heart measurement of the infant. The integrated monitoring system 10 may also include a pulse oximeter 122 communicatively coupled to the incubator monitor 30 to transmit data of a blood oxygen level of the infant. Additionally, the integrated monitoring system 10 may include a body temperature sensor 124 communicatively coupled to the incubator monitor 30 to transmit data of a body temperature of the infant. The incubator monitor 30 may therefore receive the measurements of the clinical variables and transmit the measurements to the integrated monitoring device 40 in real-time for analysis. The integration with the incubator monitor 30 and the sensors coupled to the incubator monitor 30 thus allows the integrated monitoring system 10 to leverage more clinical variables than those that may have been previously manually monitored into cohesive, machine-learning-based analysis for improved patient outcomes. In other embodiments, the incubator monitor 30 may be omitted, and the sensors 120, 122, 124 may be directly communicatively coupled to the integrated monitoring device 40.

Moreover, the camera 50 of the integrated monitoring device 40 may detect color changes of the infant as a clinical variable thereof. More specifically, an amount of yellow discoloration may be sensed by the camera 50, such that the integrated monitoring device 40 may monitor any amount or degree of jaundice in the infant. The integrated monitoring system 10 may further detect an activity level of the infant based on motion captured by the camera 50, in some embodiments. In such cases, the camera 50 may be designed to capture images or video in response to detected motion. Alternatively, the camera 50 may collect and transmit data to the controller 44 of the integrated monitoring device 40 continuously or at regular intervals. The integrated monitoring system 10 may include the microphone 52 to capture audio data of the infant and, in some embodiments, one environmental condition of the incubator includes a sound level adjacent the incubator. Additionally, the microphone 52 may capture any noises made from the infant or from within the incubator. For example, if the infant cries or speaks, the sounds may be captured in the audio data transmitted to the integrated monitoring device 40.

The integrated monitoring device 40 may receive the audio data and pass it through a high-pass or band-pass filter to identify any peaks or plateaus of noise that are above a predetermined threshold level of decibels, in certain embodiments. In some embodiments, the audio data may be further attenuated to remove any constant or background noise that may otherwise obscure the relevant signals associated with a current state of the infant.

The integrated monitoring system 10 may include a gas sensing unit to detect one or more gas concentrations within the incubator. For example, the gas sensing unit 126 may measure an oxygen concentration, a carbon dioxide concentration, and/or a volatile organic compound (VOC) concentration as environmental variables associated with the incubator. In some embodiments, the gas sensing unit 126 includes an oxygen sensor 130, a carbon dioxide sensor 132, and/or a VOC sensor 134 to measure the relevant concentrations in the incubator. As one example, the VOC concentration measured by the VOC sensor 134 may be implemented to monitor a biome of the infant, which may be utilized to evaluate potential sepsis or necrotizing enterocolitis symptoms for the infant. As such, the VOC concentration may be closely evaluated by the integrated monitoring device 40 of certain embodiments, along with other sensor data, to guide personalized and predictive treatments for the infant.

The integrated monitoring system 10 may include an environmental temperature sensor 140 to detect a temperature within the incubator, a humidity sensor 142 to detect a humidity within the incubator, a flowrate sensor 144 to detect a gas flowrate within the incubator, and/or a pressure sensor 146 to detect a pressure within the incubator. Indeed, the environment for certain high-risk infants may be monitored with multiple sensors to enable the integrated monitoring system 10 to provide a more in-depth analysis of patient health and any suggested treatment adjustments. Moreover, various correlations between the multi-dimensional sensor data and patient outcomes may be determined more quickly and/or with more certainty for each additional dimension of sensor data that is monitored via the integrated monitoring system 10. Thus, a totality of various parameters may be evaluated via the integrated monitoring system 10 to better diagnose any longitudinal changes in patient health for improved clinical decision support.

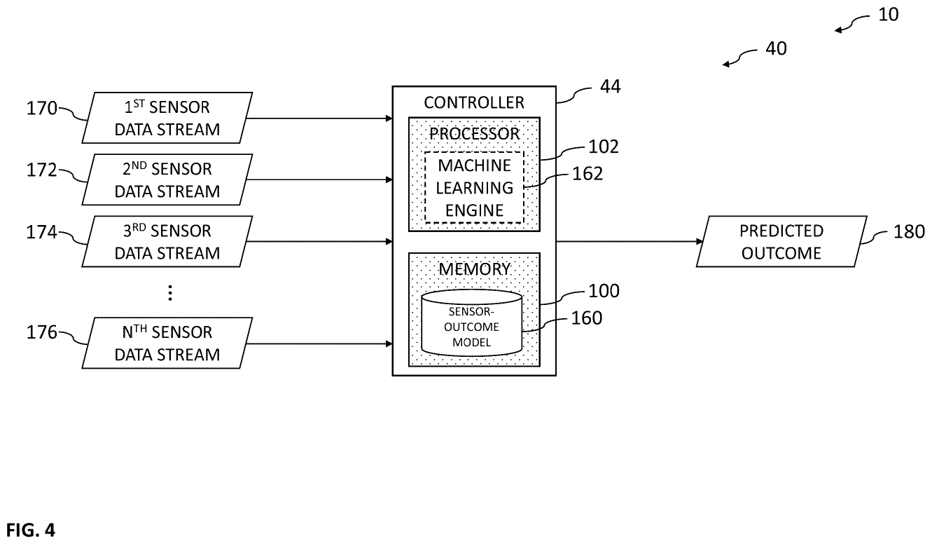

In certain embodiments, the integrated monitoring device 40, the gas sensing unit 126, and/or 30) one or more of the sensors may be provided as a kit for retrofitting existing incubators with the integrated monitoring features disclosed herein. The integrated monitoring system 10 may be installed or deployed for an existing incubator via placing or coupling the integrated monitoring device 40 on or near the cover of the incubator. The deployment may also include positioning the sensors to monitor the environmental conditions of the incubator and the clinical variables of the infant and communicatively coupling the integrated monitoring device 40 to the incubator monitor 30, the network 110, and/or the user device 56. As such, existing incubators may be upgraded with the present integrated monitoring system 10 to efficiently improve patient care. FIG. 4 is an example block diagram of the controller 44 of the integrated monitoring device 40 receiving and transmitting data during operation. As illustrated, the controller 44 may include the processor 102 and the memory 100. Moreover, the memory 100 of the controller 44 may include a sensor-outcome model 160 that is implemented to predict clinical outcomes of a patient, such as an infant, based on real-time sensor data. The sensor-outcome model 160 may be or include any suitable type and number of artificial intelligence models or machine learning algorithms. For example, certain embodiments of the sensor-outcome model 160 may include a neural network having an input node layer, one or more hidden node layers, and an output node layer. In certain embodiment, the sensor-outcome model 160 may be or include at least one convolutional neural network, long short term memory network, recurrent neural network, generative adversarial network, radial basis function network, multilayer perceptron, self-organizing map, deep belief network, restricted Boltzmann machine, autoencoder, linear regression, logistic regression, decision tree, SVM algorithm, naive bayes algorithm, KNN algorithm, k-mean, random forest algorithm, dimensionality reduction algorithm, gradient boosting algorithm, and/or combinations thereof. In some embodiments, the sensor-outcome model 160 is generated via cluster analysis of hallmark parameters or variables to link various causes with associated effects for one or more patients.

The controller 44 or the processor 102 may also include, or function as, a machine learning engine 162 that utilizes the sensor-outcome model 160. In other words, the sensor-outcome model 160 includes machine readable instructions that the processor 102 and/or the machine learning engine 162 may execute to perform the processing steps disclosed herein. Other embodiments may include the machine learning engine 162 in another portion of the controller 44, such as within the memory 100 or the sensor-outcome model 160 itself.

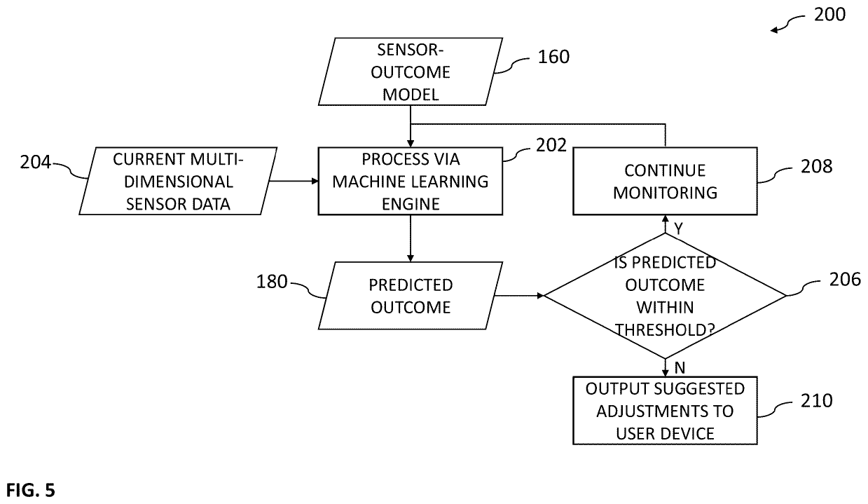

As illustrated, the controller 44 may receive multiple streams of real-time data from the sensors of the integrated monitoring system 10. For example, a 1st sensor data stream 170. 2nd sensor data stream 172. 3rd sensor data stream 174, as well as any additional data streams up to an including a Nth data stream 176 may be transmitted to the controller 44. In some embodiments, a real-time data stream may be received from each sensor of the integrated monitoring system 10. The data streams may be time synchronized in any suitable manner to facilitate consumption of relevant relationships between variables from the data streams. The controller 44 may analyze the data streams with the machine learning engine 162 applying the sensor-outcome model 160 to generate a predicted outcome 180 for the infant in the incubator. Based on predictive analysis, the controller 44 may also determine improvements or adjustments to be made to a current treatment plan that may further improve the predicted outcome 180. The operation of the integrated monitoring system 10 therefore provides comprehensive analysis of multiple sensor data types taken at a high resolution, as discussed in more detail below: FIG. 5 is a flowchart of a method 200 to use the integrated monitoring system 10 to provide clinical decision support, in accordance with aspects of the present disclosure. The method 200 may be performed by the controller 44 of the integrated monitoring device 40, in certain embodiments. As illustrated at block 202 the controller 44 (or processor 102 or machine learning engine 162 thereof) may process a sensor-outcome model 160 and current multi-dimensional sensor data 204 via a machine learning engine 162. The current multi-dimensional sensor data 204 may be an array of sensor data collected in real-time from the one or more sensors of the integrated monitoring system 10 and time synchronized based on associated metadata or timestamps. As such, this data represents a detailed picture of a current state and recent occurrences within the incubator 12. The machine learning engine 162 may process the current multi-dimensional sensor data 204 by inputting the data into the sensor-outcome model 160 to generate a predicted outcome 180 associated with the current treatment plan or trajectory of the infant.

Indeed, in embodiments in which the sensor-outcome model 160 is a neural network model, the model may correlate an input node to one or more hidden node layers, which are in turn correlated with an output node layer. The current multi-dimensional sensor data 204 may therefore be processed via node-based calculations that weight different parameters based on their importance, and generally output one or more values indicative of an expected outcome for the infant. For example, various weights of the sensor-outcome model 160 may assign higher importance or relevance to a body temperature of the infant, than to an environmental temperature of the incubator 12. As another example, the concentration of oxygen may be more relevant to a current treatment plan than a concentration of carbon dioxide. In some embodiments, the weights of the neural network may be individually tailored or trained for the specific infant being treated.

At block 206, the controller may determine if the predicted outcome 180 is within a threshold. In certain embodiments, the threshold may be a numerical value, such as a probability, that is associated with certain outcomes. As such, one or more thresholds may be set to facilitate monitoring of whether the probability of an undesired outcome, symptom, or diagnosis occurring is above an acceptable level. Certain embodiments may also include thresholds for a rate of change of a variable associated with the infant and/or an acceleration, deceleration, or inflection point of a variable associated with the infant. At block 208, if the predicted outcome 180 is within a threshold, the controller may continue monitoring the infant by receiving additional sensor data and repeating previous determinations of the method 200. For example, the predicted outcome 180 of certain embodiments may indicate that the current treatment plan is already optimized to improve or maintain the health of the infant.

At block 210, if the predicted outcome 180 is not within a threshold, the controller 44 may output one or more suggested adjustments to the user device 56. In such embodiments, the predicted outcome 180 may be a physical state that indicates the current treatment plan is less than optimal. For example, continuing treatment or care without change may be predicted to cause a certain condition or symptom for the infant. The controller 44 may also identify a more optimal treatment to reduce symptoms based on the sensor-outcome model 160. The controller 44 may therefore implement the relationships of the neural network to determine one or more suggested improvements to improve the current physical state of the infant. The controller 44 may also provide an indication of the suggested improvements to the user device 56 for a healthcare provider to implement.

In some embodiments, the controller 44 may additionally or alternatively present, via the user device 56, a request for authorization to automatically implement the suggested improvements. As such, the healthcare provider may authorize the controller 44 of the integrated monitoring system 10 to make the changes automatically. In some embodiments, the healthcare provider may further provide authorization for the controller 44 to automatically implement changes for a predetermined time period, such as a certain number of minutes or hours, until a specified symptom is resolved, until the infant is released from the incubator 12, and so forth. In certain embodiments, the controller 44 may further compare the predicted outcome 180 to a second, higher threshold representing a bar for initiating an escalated response. For example, if the infant has a certain significantly decreased or decreasing clinical variable, the controller 44 may proceed to instruct subsystems of the incubator to implement treatment adjustments for immediate infant care.

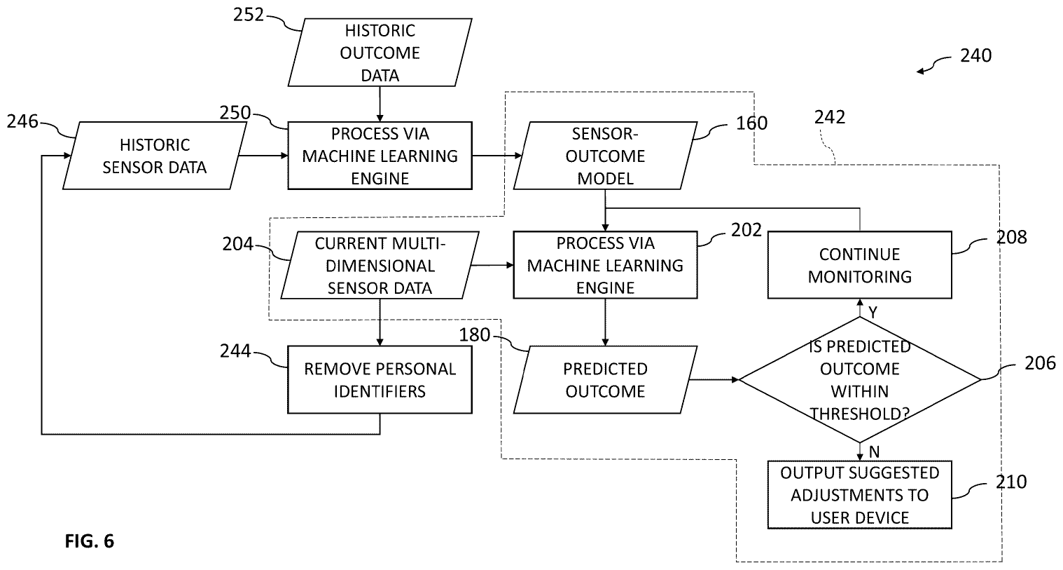

FIG. 6 is a flowchart of a method 240 for further training the sensor-outcome model of the integrated monitoring system 10 based on the current multi-dimensional sensor data 204, in accordance with aspects of the present disclosure. The method 240 may be performed by the controller 44 of the integrated monitoring device 40, in certain embodiments. Moreover, an outline 242 is provided to indicate elements of method 240 that may generally correspond to previously introduced elements of method 200. For improved clarity, the description of these elements is not repeated here.

Generally, the method 240) illustrates how the regular operational data of the integrated monitoring system 10 may be fed back into the sensor-outcome model 160 for further refinement thereof. For example, the current multi-dimensional sensor data 204 may be processed to determine the predicted outcome 180, as introduced above. As illustrated in block 244, the controller 44 may also remove one or more personal identifiers from the current multi-dimensional sensor data 204 to generate historic sensor data 246 or scrubbed sensor data. For example, the controller 44 may anonymize the data by removing references to a name of the infant, and instead associate the data with an alternative name or reference number. The data may also be cleansed for any other identifying information, such as birthdate, home address, contact information, and so forth.

As illustrated at block 250, the controller 44 (or processor 102 or machine learning engine 162 thereof) may process historic sensor data 246 and historic outcome data 252 via the machine learning engine 162 to generate the sensor-outcome model 160 or improvements thereto. In some embodiments, the historic outcome data 252 includes one or more diagnoses or health evaluations the infant received during or after collection of the corresponding historic sensor data 246. The historic outcome data 252 may therefore serve as “labels” to the corresponding historic sensor data 246, guiding the training of the sensor-outcome model 160 correlations and weights. As an example, if the infant was diagnosed with mild anemia or jaundice during a stay in the incubator, that information may be tagged or appended to the concurrent sensor data by a healthcare provider so that the sensor-outcome model 160 can discern certain relationships between the environmental conditions, clinical variables, and heath conditions for later use. After further training is complete, the updated sensor-outcome model 160 may also be provided to additional integrated monitoring devices 40 so that other infants may benefit from the information gleaned during a previous infant's treatment. In such embodiments, the other integrated monitoring devices 40 may receive the updated sensor-outcome model 160 from a secure web application, from another suitable wireless device, or from a physical storage device. The sensor-outcome model 160 leveraged by the integrated monitoring system 10 disclosed herein may therefore be constantly updated based on an increasing database of historic sensor data 246 that does not comprise any personal information of infants.

The integrated monitoring system 10 disclosed herein may be provided with any suitable format or arrangement of components that facilitates predictively directing patient health toward optimal outcomes based on monitoring a set of real-time sensor data streams. For example, some or all of the above features discussed with reference to the integrated monitoring system or integrated monitoring device 40 may be incorporated into one or more modules. In certain embodiments, each module of the integrated monitoring system 10 may perform one or more respective functions, thus enabling one or more components, processors, or programs to be specialized or specifically adapted to the respective functions.

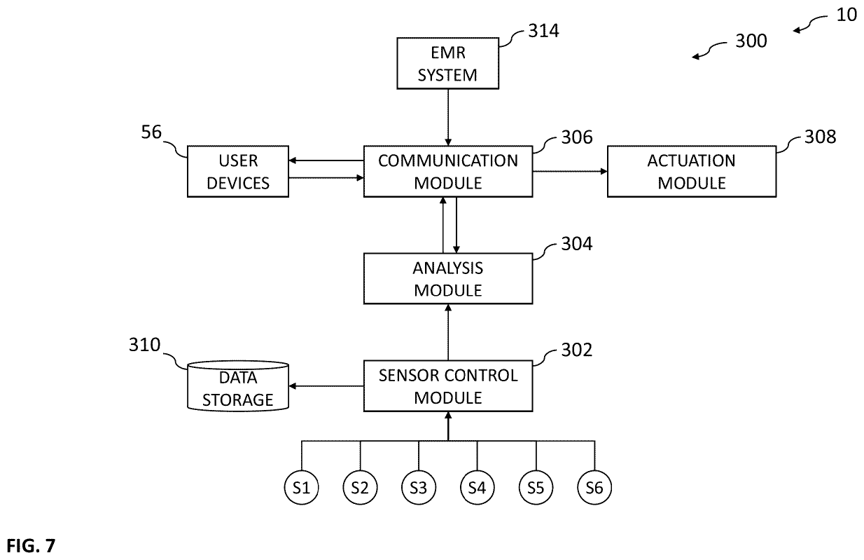

FIG. 7 illustrates an example of an integrated monitoring system 10 for monitoring a patient in an incubator 12, in accordance with aspects of the present disclosure. In the illustrated embodiment, the integrated monitoring system 10 includes a modular system 300 having multiple sensors (such as sensors S1-S6), a sensor control module 302, an analysis module 304, a communication module 306, and an actuation module 308. The modules may each perform a respective function within the integrated monitoring system 10, where the functions cooperate or interleave with one another to facilitate improved patient monitoring. It should be understood that the modules and sensors may be combined or arranged in any suitable manner that facilitates the present techniques.

While the integrated monitoring system 10 of FIG. 7 may be described in terms of its use with a single incubator 12, it is understood that the integrated monitoring system 10 may be used with more than one incubator 12. For example, in one implementation, a single sensor control module 302 may control sensors attached to multiple incubators 12 and a single actuation module 308 may control parameters associated with the multiple incubators 12. Moreover, certain components or modules of the integrated monitoring system 10 may be positioned within a shared housing or any suitable number of individual housings. As an example, the sensor control module 302 may be positioned in a first location near the incubator 12, the analysis module 304 and communication module 306 may be positioned together and remote from the incubator 12, and the actuation module 308 may be positioned in a second location near the 30) incubator 12. Another example embodiment includes each module being provided within a single computer or controller having one or more processors and memories. Indeed, the modular nature of embodiments of the integrated monitoring system 10 facilitates distribution and scaling of the modules for monitoring any number of incubators, in any suitable physical form factors.

In an embodiment, any desired number of sensors S1-S6 may be distributed within or around the incubator 12 and the surrounding environment to sense and collect real-time data therefrom. For example, the sensors S1-S6 may include environmental sensors that monitor the environment inside and/or outside of the incubator 12 and clinical sensors that monitor the variables indicative of a status of the infant inside the incubator 12. As discussed above, certain sensors S1-S6 may monitor one or more environmental conditions of the incubator 12, such as a particular gas composition, an environmental temperature, a humidity, a flowrate, and/or a pressure, and/or may monitor one or more clinical variables of the infant, such as a temperature, a blood measurement, and/or a heart measurement.

As further detailed examples of the sensors S1-S6, environmental sensors thereof may include, for example, any suitable combination of temperature sensors: humidity sensors: pressure sensors for measuring ambient air pressure; flow sensors for measuring the flow of gasses in and out of the incubator; gas sensors for measuring the ambient concentrations of oxygen, carbon dioxide, VOCs, and other gasses; and light sensors for measuring the intensity and wavelengths of light in the incubator. The clinical sensors may include, for example, any suitable combination of temperature sensors for measuring body temperature, pulse oximeters, EKG sensors, and respiration sensors. The environmental and clinical sensors may also include cameras and microphones for measuring the infant's clinical status including, for example, activity level, pain level, and the color of skin, sclera, and other body parts. Collectively, these sensors S1-S6 may provide real-time, individualized data collection and monitoring of the status of the patient via the integrated monitoring system 10 disclosed herein. Certain examples of these sensors S1-S6 are discussed above with reference to FIG. 3.

The sensor control module 302 of certain embodiments may operate the sensors S1-S6 and collect sensor data therefrom. The sensor control module 302 may be communicatively coupled between the sensors S1-S6 and the analysis module 304, in some embodiments. Additionally, a data storage 310 may be communicatively coupled to the sensor control module 302 to store the sensor data from the sensors S1-S6, in some embodiments. The sensor control module 302 may also store the recorded sensor data for offline analysis, such as the training of machine learning (ML) models and/or updating of the sensor-outcome model 160 discussed above. The trained ML models may then be implemented in the analysis module 304 for use in real-time monitoring of the patient. In some embodiments, the sensor control module 302 may thus perform all or a portion of the operations of the integrated monitoring device 40 and/or the sensor hub 80 discussed above with reference to FIGS. 1 and 2.

In embodiments, the analysis module 304 analyzes the sensor data, as well as any other data (such electronic medical record data) obtained from the communication module 306, to evaluate patient status and the correctness or accuracy of the treatment conditions. As such, the analysis module 304 may be communicatively coupled between the sensor control module 302 and the communication module 306. The analysis module 304 may use trained machine learning models to assess patient status and make treatment recommendations. For example, the analysis module 304 may compute the likelihood of particular outcomes, for example, a healthy outcome or a certain condition such as retinopathy of prematurity, given the current status and treatment. The analysis module 304 may also recommend changes to the treatment to improve outcomes. For example, the analysis module 304 may recommend increasing or decreasing the oxygen concentration, adjusting the light intensity or wavelength, increasing or decreasing the humidity, and so forth. In embodiments, the analysis module 304 of certain embodiments may include some or all of the functionality described above with reference to the integrated monitoring device 40 or controller 44 thereof, such as the sensor-outcome model 160 to facilitate analysis and prediction of clinical outcomes of a patient based on real-time sensor data.

The communication module 306 of the integrated monitoring system 10 may be communicatively coupled between the analysis module 304 and other components of the integrated monitoring system 10. The communication module 306 may be connected to the other components via any suitable wired or wireless communication protocols. As an example, the communication module 306 of some embodiments may provide alerts to user devices 56, such as staff devices associated with clinical staff or healthcare providers. The alerts transmitted by the communication module 306 may be constructed to include any patient status that is or is predicted to exceed a threshold probability, including current or expected complications. The communication module 306 may also provide alerts to notify the staff about recommended changes to the treatment, as developed by the analysis module 304. In some cases, the communication module 306 may request approval from staff before implementing the recommended changes to the treatment. In certain cases, the integrated monitoring system 10 may directly implement the changes. Additionally, the communication module 306 may also provide an ability for treatment changes to be initiated by the staff. The communication module 306 may also exchange data with other data systems, such as an electronic medical record (EMR) system 314. For example, the communication module 306 may obtain information about the patient's medical history and treatment plan from the EMR system 314, which may be relayed to the analysis module 304 for evaluation of patient status.

The actuation module 308 receives commands from the communication module 306 and implements the treatment changes by changing the operating conditions of the incubator 12. In embodiments, the actuation module 308 includes or is communicatively coupled between the communication module 306 and one or more actuators or control devices that may cause changes in operating conditions based on receiving signals from the actuation module 308. For example, the actuation module 308 may change one or more of the temperature, oxygen levels, humidity levels, gas flow rates, light intensity, light wavelength, and so forth, associated with the incubator being monitored. In some embodiments, indications of any types of changes that the integrated monitoring system 10 or the actuation module 308 thereof is authorized to perform directly may be provided by the user devices 56. For example, certain integrated monitoring systems 10 may be preauthorized to automatically adjust one or more environmental conditions of the incubator 12, based on prior approval of the type of adjustments by a clinician.

Accordingly, embodiments of the integrated monitoring system 10 may provide decision support to medical providers, generate optimal treatment plans, recognize early signs of potential complications, and/or automatically regulate incubator conditions and set points to ensure optimal outcomes for the infants. As such, the integrated monitoring system thus provides enhanced monitoring and/or treatment of infants within incubators

The present disclosure described herein, therefore, is well adapted to carry out the objects and attain the ends and advantages mentioned, as well as others inherent therein. While a presently preferred embodiment of the disclosure has been given for purposes of disclosure, numerous changes exist in the details of procedures for accomplishing the desired results. These and other similar modifications will readily suggest themselves to those skilled in the art and are intended to be encompassed within the spirit of the present disclosure disclosed herein and the scope of the appended claims.

What is claimed is:

1. An integrated monitoring system for supporting an infant in an incubator, comprising:
a plurality of sensors configured to sense at least one environmental condition of the incubator and at least one clinical variable of the infant;
an integrated monitoring device communicatively coupled to the plurality of sensors, the integrated monitoring device comprising a processor and a non-transitory machine-readable storage medium storing processor-executable instructions that, when executed by the processor, cause the processor to:
receive the at least one environmental condition of the incubator and the at least one clinical variable of the infant from the plurality of sensors;
generate a predicted outcome based on application of the at least one environmental condition of the incubator and the at least one clinical variable of the infant to a model configured to associate sensor data with clinical outcomes; and
in response to the predicted outcome being outside a threshold, output a notification to a user device communicatively coupled to the integrated monitoring device.

2. The integrated monitoring system of claim 1, wherein the non-transitory machine-readable storage medium includes executable instructions, when executed by the processor, to cause the processor to receive the at least one environmental condition of the incubator and the at least one clinical variable of the infant in real-time as a continuous data stream.

3. The integrated monitoring system of claim 1, wherein the non-transitory machine-readable storage medium includes executable instructions, when executed by the processor, to cause the processor to generate the model by analyzing historic sensor data and historic outcome data with a machine learning engine, and wherein the historic outcome data comprises a tag labeling an associated portion of the historic sensor data with at least one diagnosis of a previous patient.

4. The integrated monitoring system of claim 1, wherein the non-transitory machine-readable storage medium includes executable instructions, when executed by the processor, to cause the processor to further train the model based on the at least one environmental condition of the incubator, the at least one clinical variable of the infant, and at least one associated diagnosis.

5. The integrated monitoring system of claim 1, wherein the threshold is indicative of a range of predicted physical states equal to or improved relative to a current physical state of the infant.

6. The integrated monitoring system of claim 1, wherein the incubator comprises a first incubator, and wherein the integrated monitoring system comprises:
a first sensor hub attached to the first incubator and communicatively coupled to the integrated monitoring device to transmit first sensor data from the plurality of sensors thereto; and
a second sensor hub attached to a second incubator and communicatively coupled to the integrated monitoring device to transmit second sensor data from an additional plurality of sensors thereto, wherein the integrated monitoring device is configured to transmit one or more notifications to the user device based on analysis of the first sensor data and the second sensor data.

7. The integrated monitoring system of claim 1, comprising an incubator monitor physically coupled to the incubator and communicatively coupled to the integrated monitoring device.

8. The integrated monitoring system of claim 7, comprising an electrocardiogram sensor communicatively coupled to the incubator monitor, wherein the at least one clinical variable of the infant comprises a heart measurement of the infant.

9. The integrated monitoring system of claim 7, comprising a pulse oximeter communicatively coupled to the incubator monitor, wherein the at least one clinical variable of the infant comprises a blood oxygen level of the infant.

10. The integrated monitoring system of claim 7, comprising a body temperature sensor communicatively coupled to the incubator monitor, wherein the at least one clinical variable of the infant comprises a body temperature of the infant.

11. The integrated monitoring system of claim 1, wherein the plurality of sensors comprises a camera configured to capture image data of the infant, and wherein the at least one clinical variable of the infant comprises an amount of yellow discoloration.

12. The integrated monitoring system of claim 1, wherein the plurality of sensors comprises a microphone configured to capture audio data of the infant, and wherein the at least one environmental condition of the incubator comprises a sound level adjacent the incubator.

13. The integrated monitoring system of claim 1, wherein the plurality of sensors comprises a gas sensing unit configured to detect one or more gas concentrations within the incubator, and wherein the at least one environmental condition of the incubator comprises one or more of an oxygen concentration, a carbon dioxide concentration, or a volatile organic compound concentration.

14. The integrated monitoring system of claim 1, wherein the plurality of sensors comprises an environmental temperature sensor configured to detect a temperature within the incubator, and wherein the at least one environmental condition of the incubator comprises the temperature within the incubator.

15. The integrated monitoring system of claim 1, wherein the plurality of sensors comprises a humidity sensor configured to detect a humidity within the incubator, and wherein the at least one environmental condition of the incubator comprises the humidity within the incubator.

16. The integrated monitoring system of claim 1, wherein the plurality of sensors comprises a flowrate sensor configured to detect a gas flowrate within the incubator, and wherein the at least one environmental condition of the incubator comprises the gas flowrate within the incubator.

17. The integrated monitoring system of claim 1, wherein the plurality of sensors comprises a pressure sensor configured to detect a pressure within the incubator, and wherein the at least one environmental condition of the incubator comprises the pressure within the incubator.

18. An integrated monitoring device for supporting a patient in an incubator, comprising:
a microphone configured to collect audio data associated with the incubator;
a camera configured to collect video data associated with the incubator;
a plurality of input/output ports configured to connect to a plurality of sensors to collect sensor data associated with the incubator; and
a controller comprising a processor and a non-transitory machine-readable storage medium storing processor-executable instructions that, when executed by the processor, cause the processor to:
receive the audio data, the video data, and the sensor data;
combine the audio data, the video data, and the sensor data into a stream of multi-dimensional sensor data;
process the stream of multi-dimensional sensor data through a sensor-outcome model configured to associate past sensor data with clinical outcomes to generate a predicted outcome; and
in response to determining that the predicted outcome is outside a threshold, output a suggested treatment adjustment to a user device.

19. The integrated monitoring device of claim 18, wherein the sensor-outcome model is stored locally within the non-transitory machine-readable storage medium.

20. The integrated monitoring device of claim 18, comprising a networking component communicatively coupled to a cloud network configured to remotely store the sensor-outcome model.

21. The integrated monitoring device of claim 18, wherein the stream of multi-dimensional sensor data comprises at least one environmental condition of the incubator and at least one clinical variable of the patient.

22. The integrated monitoring device of claim 18, comprising a housing configured to fasten to the incubator and physically support the microphone, the camera, the plurality of input/output ports, and the controller.

23. The integrated monitoring device of claim 18, wherein the plurality of input/output ports is configured to communicatively couple to an incubator monitor mounted to a body of the incubator.

24. The integrated monitoring device of claim 18, wherein the plurality of input/output ports is configured to communicatively couple to a gas sensing unit configured to detect one or more of an oxygen concentration, a carbon dioxide concentration, or a volatile organic compound concentration within the incubator.

25. The integrated monitoring device of claim 18, wherein the plurality of input/output ports is configured to communicatively couple to an environmental temperature sensor, a humidity sensor, a flowrate sensor, a pressure sensor, or a combination thereof.

26. A method of providing clinical decision support for monitoring a patient of an incubator, the method comprising:
receiving, via a processor of an integrated monitoring device:
audio data from a microphone of the integrated monitoring device;
video data from a camera of the integrated monitoring device; and
sensor data from a plurality of sensors associated with the integrated monitoring device;
combining, via the processor, the audio data, the video data, and the sensor data into a stream of current multi-dimensional sensor data
processing, via the processor, the stream of current multi-dimensional sensor data through a sensor-outcome model to generate a predicted outcome; and
in response to a determination that the predicted outcome is outside a threshold, outputting a suggested treatment adjustment to a user device.

27. The method of claim 26, wherein the suggested treatment adjustment comprises adjusting an incubator temperature, an incubator humidity, an incubator gas flow, an incubator pressure, an incubator light level, an incubator sound level, or a combination thereof.

28. The method of claim 26, comprising:
receiving, via the processor, an indication of clinician approval of the suggested treatment adjustment; and
authorizing, via the processor, a subsystem of the incubator to perform the suggested treatment adjustment.

29. The method of claim 26, wherein the user device comprises a workstation computer associated with the incubator or a mobile device of a clinician associated with the patient.
